(19) United States
(12) Patent Application Publication
(10) Pub. No.: US 2019/0054261 A1
(43) Pub. Date: Feb. 21, 2019

(54) VAPOR MEDICINE DISPENSING AND NANO-MIST LUNG CLEANSER SYSTEM WITH FACE ADAPTER

(71) Applicant: Nano Mist, LLC, Los Angeles, CA (US)

(72) Inventor: Isaac Banoun, Los Angeles, CA (US)

(21) Appl. No.: 16/166,664

(22) Filed: Oct. 22, 2018

Related U.S. Application Data
(63) Continuation-in-part of application No. 15/422,872, filed on Feb. 2, 2017.

(51) Int. Cl. A61M 15/08; A61K 9/00; A61M 16/10; A61M 15/00; A61M 16/06; A61M 39/24

(52) U.S. Cl. CPC A61M 15/08; A61K 9/0078; A61M 16/109; A61M 15/0016; A61K 31/045; A61K 9/008; A61M 39/24; A61K 9/0075; A61M 16/06

(57) ABSTRACT

A system, accompanying apparatuses and methods relating to an oral or nasal enhanced nano-mist delivery and lung cleansing system having for an object to provide a safe system and method of delivering heated, moist, flavored air into the lower TB and alveolar regions of lungs.

### CROSS REFERENCE TO RELATED APPLICATION

This application is a continuation-in-part and claims the benefit of and takes priority from U.S. patent application Ser. No. 15/422,872 filed on Feb. 2, 2017, the contents of which are herein incorporated by reference.

### FIELD OF THE INVENTION

The present system relates to medical systems for delivery of drugs, medications, cleansers, heated, moist, or flavored air.

### BACKGROUND OF THE INVENTION

Drug delivery refers to approaches, formulations, technologies, and systems for transporting a pharmaceutical compound in the body as needed to safely achieve its desired therapeutic effect. It may involve scientific site-targeting within the body or facilitating systemic pharmacokinetics; in any case, drug delivery is typically concerned with both quantity and duration of drug presence. Drug delivery is often approached via a drug's chemical formulation, but it may also involve medical devices or drug-device combination products. Drug delivery is a concept heavily integrated with dosage form and route of administration, the latter sometimes even being considered part of the definition. Concurrent drug delivery technologies modify drug release profile, absorption, distribution and elimination for the benefit of improving product efficacy and safety, as well as patient convenience and compliance. Drug release is achieved by diffusion, degradation, swelling, and affinity-based mechanisms. Most common routes of administration include the preferred non-invasive perioral (through the mouth), topical (skin), trans mucosal (nasal, buccal/sublingual, vaginal, ocular and rectal) and inhalation.

Many medications such as peptide and protein, antibody, vaccine and gene-based drugs may not be delivered using these routes because they are susceptible to enzymatic degradation or cannot be sufficiently absorbed into the systemic circulation efficiently due to molecular size and charge issues. For this reason, many protein and peptide drugs have to be delivered by injection or a nano-needle array.

For example, many immunizations are based on the delivery of protein drugs and are often done by injection. Current efforts in the area of drug delivery include the development of targeted delivery in which the drug is only active in the target area of the body (for example, in cancerous tissues) and sustained release formulations in which the drug is released over a period of time in a controlled manner from a formulation. In order to achieve efficient targeted delivery, the designed system must avoid the host's defense mechanisms and circulate to its intended site of action. Types of sustained release formulations include liposomes, drug loaded biodegradable microspheres, and drug polymer conjugates.

Concurrently, delivery through oral means has been shown to be very effective, particularly in pharmaceutical aerosols which may be delivered to the lungs through oral inhalation using such mechanisms as nebulizers, mist inhalers, metered dose inhalers and dry powder inhalers. Compared with oral inhalation, it has been found that the administration of pharmaceutical aerosols through the nose to the lungs is advantageous in a number of situations. Delivery via the nose, directly to the lung of such substance as aerosols may improve patient comfort and compliance if a simple cannula can be used for medications with long delivery times as well as those that require frequent dosing.

While a number of advantages exist for nose-to-lung delivery of pharmaceutical aerosols, one of the major draw backs to nasal delivery of numerous substances focuses on the fact that the nose is basically design to include a very effective filtration for particles, namely cilia of the two nasal cavities.

The two nasal cavities condition the air to be received by the other areas of the respiratory tract. Owing to the large surface area provided by the nasal conchae (also known as turbinates), the air passing through the nasal cavity is warmed or cooled to within 1 degree of body temperature. In addition, the air is humidified, and dust and other particulate matter is removed by vibrissae, short, thick hairs, present in the vestibule. The entire mucosa of the nasal fossae is covered by a blanket of mucus, which lies superficial to the microscopic cilia and also filters inspired air. The cilia of the respiratory epithelium move the secreted mucus and particulate matter posteriorly towards the pharynx where it passes into the esophagus and is digested in the stomach. The nasal cavity also houses the sense of smell and contributes greatly to taste sensation through its posterior communication with the mouth via the choanae.

Numerous recent studies have indicated that delivery of certain applicable substances through nasal cavities has proved more advantageous than through conventional oral intake methods. Thus, the directive includes ensuring nose-to-lung delivery with streamlined nasal cannulas and condensational aerosols was highly efficient and targeted deposition to the lower TB and alveolar regions.

### SUMMARY OF THE INVENTION

The instant apparatus, system and accompanying methods, as illustrated herein, is clearly not anticipated, rendered obvious, or even present in any of the prior art mechanisms, either alone or in any combination thereof. Therefore, it is an object of the instant invention to provide various systems and accompanying apparatuses for oral, nasal or propellant mist enhanced delivery and lung cleansing system and associated apparatuses.

The present system, accompanying apparatuses and methods relate to an oral, nasal or propellant enhanced mist delivery and lung cleansing apparatuses and has for an object to provide a safe and harmless means for and method of delivering medication with heated, moist, flavored air; or by inhaling warm medication like methanol into the lungs in case of a respiratory ailment under direction of a physician.

It is an additional objective to introduce a system wherein nano-particles are generated wherein the nano-particles are of a size and order to not become trapped and filtered by the cilia and thus possess an ability to reach the alveoli and lung sacs.

It is an additional objective to introduce a system wherein depositional losses of drug dosages in the nose are eliminated through the production and utilization of smaller than conventional sized pharmaceutical aerosols (3-7 μm) particles.

In contrast with conventional delivery, the use of initially submicrometer aerosols and condensational growth techniques have recently been shown to provide very efficient nose-to-lung drug delivery

It is an additional objective to introduce a system utilizing Nano-Mist which is a concentration of extremely small measured droplets in a size range from 50 nano-meters to 400 nano-meters.

It is a further object to introduce a nose-to-lung delivery approach with targeted deposition to the lower TB and alveolar regions.

It is a further object to introduce a system including a face mask which forms an occlusive seal with an intake portion of human face in order not to minimize loss of nano-particles.

It is also an object to introduce a system including a face mask which forms an occlusive seal with the area surrounding the human nose in order not maximize intake and mimimize loss of nano-particles.

It is also an object to introduce a system including a face mask which forms an occlusive seal with the area surrounding the human mouth in order not maximize intake and minimize loss of nano-particles.

It is also an objective to introduce a system including a primary use of life extension and pain management. Another object of the system is to provide an article of manufacture wherein air may be drawn through a porous substance or filter mechanism of a cartridge which has been moistened with a chemically harmless flavoring preparation.

An additional objective of the instant system is to combine moisture and taste wherein the moist and flavored air passes through a section of the device heated by a suitable heating element in order that warm, moist and flavored air is drawn into the mouth of the user, and if desired, into the lungs of the user.

A further object of the instant system is to provide a nano-vaporization system for circulating the fluid around the heating element in a turbulent manner to suitably raise the temperature of the inhalant mixture, with the purpose that the temperature of the flavored air.

A further object is to insulate the heat source as the mechanism may be too hot for the user to hold as well as to introduce a nasal adapter device to be utilized with instant vapor medicine delivery system, which can be adapted for both human and animal applications. An additional object is to introduce a nano-misting or nano-mist system, which can be adapted for both human and animal applications.

A further object of the instant system is to introduce an apparatus with the capability of delivering water, menthol, eucalyptus or glycerol beads of a micro size when compared to current vaporizers by Vicks® or other such systems which deliver large water drops that don't go deep in the lungs. Moreover, concurrent inhalers and nebulizers do not deliver droplets measured in nanometers.

An added and prominent objective of the instant device is to introduce a face mask to prevent transmission of disease and a filtration system to protect against potential contamination. And, an unexpected result of experimentation with drug delivery processes focuses on the positive utilization of the by-product vapor alone to clean, clear and soothe the lungs and enhance lung functions with the clean steam with small particle size.

An unexpected result of vaporizers is that the vapor can carry beneficial drugs that actually treat diseases as well as administer treatments and therapies. Unlike today's existing technology which is used as a cigarette cessation or replacement product containing the carcinogen nicotine and in no way is it used or meant to be for the benefits, promotion of health in fact curing or pain managements here cited. You are in effect steam cleaning your lungs and thus entails a whole new entry point for these drugs into the body. Also introduced, a plain water vapor, with nothing added, from these devices, was observed to open the lungs and allow the user to breathe better.

The varied the size of vapor droplets to suit the molecule size of drugs using the existing technology heat control can lead to immediate migraine treatment with such drugs Sumitriptan®. It was not expected that existing devices can work for headaches and other pain management, such as NSAIDs for arthritis.

In additional embodiments, the system may be design to utilize pharmaceutical prescribed drugs (other not nicotine) and caffeine may be help reduce the boiling point or volatilization of another drug. It can also be used for energy vapor markets to replace the energy drink space. Unexpected was that these devices deposit drugs under tongue and in all mouth receptors so delivery is not only through lungs. Different than current use which is intended for lung delivery.

Further addressing the nasal vaporizing as another point of entry totally different than concurrent systems, this would be beneficial to treat many things nasally as well as another entry point to the lungs that is not done using current tech. Also, replaces nasal spray.

Intranasal is a good area for low dose potent drugs, since surface area is normally too small. An additional route for vaccines since you need very little to obtain an antigenic immune response. Thus the instant system allows the user to administer gene therapy as well as viruses and vaccines through the lungs.

Additionally, the system may be utilized for diagnostics and may deliver small molecule drugs as deep as this into the lungs. The depth and reach of drug deposition is unique as the stomach is bypassed stomach for efficacy, purity and speed and thus the drugs or medicines are inherently purer. Thus, mixtures or cocktails can be blended. For example, adding caffeine for energy and to lower the vaporizing temp for other drugs that will not survive the heat.

Aromatherapy usage can clearly provide more focused attention and can be part of our nutritional or neutraceuticals. Wine and alcohol mixed with flavors and glycol or other carriers. Any and all compounds and mixtures may be utilized to cleanse lungs, including common menthol. Sublingual, and mouth and lung combination absorption is possible, including B12 absorption. The vaped B12 will be absorbed thru the mouth and the lungs providing extra energy to the body.

Further, nacetyl lcysteine may be vaped into the lungs and combining this with a nonionic surfactant like polysorbate 80 (TWEEN 80) to create the best available compound which will also be mucolytic. Additives for infections may also be utilized. Thus, all nutraceuticals may be employed, again because the process is in liquid form. And a further combination of mucolytic drugs, cysteine and a non-ionic surfactant (TWEEN 80, poysorbate or other non-ionic surfactants can also be used. The system can therefore replace nasal spray because it delivers a micro-aerosolized dose of therapeutic medication.

Thus, the instant system to deliver drugs and other actives via various routes where other delivery systems have failed or require injections. The delivery system allows drugs to be vaporized (vaped) and efficiently delivered to the lungs, through the nasal route and mouth, including sublingual and across other mucosal membranes. The target market includes delivering efficiently nutraceuticals, herbals, prescription and non-prescription drugs including vitamins, vaccines, anti-pyretics, pain (narcotic and non-narcotic including migraine headaches), diabetes, cystic fibrosis, drugs for obesity and anti-ageing, in a vaporized state. Several patents have been filed to include over-the-counter (OTC) drugs, nutraceuticals, prescription drugs such as nitroglycerin, sildenafil (Viagra, and other similar drugs), active ingredients of Vicks VapoRub and recreational and lifestyle style improving drugs. All drugs in a solvent system that can be vaporized and are heat stable under the E-cig temperatures are included. Vaccines can be delivered in developing countries in a cost-effective manner without cross contamination by changing the disposable tip attached to the device.

Additionally, in one embodiment, the instant device will be unique for medicine as it will comprise a one-way fill valve and thus operate as a one way inhaler to prevent spread of disease if device is being shared, and HEPA® and other filters or better. The fill valve will be spring-loaded may be torsion or other such mechanism as well and which allows the cartridge to be filled with a “needle-point” bottle. The cartridge may be a disposable tip with a one way valve.

For the instant system, this will allow for the cartridge to be filled with medication, while preventing the “patient” from extracting the solution from the cartridge. A specific voltage may to be preset on the battery device specific for each medication, along with an automatic shut-off feature preventing the solution from reaching a defined maximum temperature.

In additional embodiments, steroids, testosterone alcohol, cortisone, prednisone may be administered. Further and even blowing it out in a fine mist to cure large areas of the outside of the body. Can administer burn and many therapies or actual drugs like Botox. Furthermore, a vaporizing vaccine is introduced and also the clearance of lungs due to Propylene Glycol, using Camphor in small particle emission and even vaping of antibiotics to treat pneumonia rather quickly, and a process for treating lung disease with herbs.

The instant system can thus transport antitussive herbs to reduce respiratory spasms; expectorant herbs loosen mucus; demulcent herbs sooth irritated tissue; and antimicrobial herbs resolve infections. For transportation of drugs, in the instant system they may not have to be frozen as they are submerged in the VG/PG or other carrier, thus buried and protected. Also process patents for the drugs that have never been processed in this manner. The drugs are processed through the lungs, not IV, Pills, Powder inhalers, or nebulizers. The system also incorporates sublingual administration as the vapor effects all receptors of the mouth.

Moreover, many processes for the treating of disease and introducing drugs are herein illustrated, for example the system may be utilized to assist breathing for asthma patients. Further, in any of the mixture, the addition of a small quantity of water in the mix to, can prevent dehydration a recurring occurrence with VG/propylene glycol non-aqueous systems. The quantity of the water will be adjusted so that we do not get precipitation of the active. If required, additional ingredients will be added such that the system delivers with a clear mixture or a colored mixture as required for medical or diagnosis purposes. Thus, the instant system represents discovered a new way to improve the current VG/PG mix and transport of all known drugs using an enveloping solution like PG/VG.

Further, Lavender oil, which possesses a scent with a calming effect which may aid in relaxation and the reduction of anxiety and stress may be employed. Lasea capsules containing Lavender oil with a high amount of linalool and linayl acetate termed as Silexan by the manufacturer are approved as an anxiolytic in Germany. The approval is based on the finding that the capsules are comparable in effect to low-dose lorazepam. Additionally, all of the systems herein are adaptable to veterinary use.

Moreover, the instant system may cover all parenteral and non-parenteral routes for administering drugs. The parenteral routes include intravenous, intramuscular, subcutaneous and intradermal. The non-parenteral routes include oral, sublingual, buccal, nasal and vaginal. Drugs such as Bayer®, baby aspirin, and other drugs for migraine and headache, as well as, Pfizer® Viagra® when delivered sublingually map provide biological stability and rapid absorption of the drug.

Yet another object of the present system is to provide a lung cleansing treatment that cleans and clears lungs due to its depth of reach into the smallest airways. Lung cleansing treatment works because the size of the droplet created by the current system is measured in nanometers, and droplets at this size can go to the smallest of airways in the lungs, where menthol and other ingredients inside these particles can be delivered. The droplets are able to attach themselves to the cold receptors in the lining of the airways, which creates shrinkage. The skin retracts as the menthol opens the airways, creating greater breathing capacity. The smallest size of the droplets is key for reaching areas in hard to reach places within the lungs. Most inhaled corticoid drug particles, due to their large size, only make it to half of the lungs, and most of the powder stays in the throat.

It is one objective of the system to create nano-particles are thus so small in stature as to penetrate the alveoli, and thus proceed where powdered drugs are unable. The current system generates a mist that as stated is composed of tiny droplets in the low nanometer range (menthol in a gaseous state). The menthol is soothing and cooling in a gaseous state that on inhalation reaches the deep innermost portions of the lungs.

There has thus been outlined, rather broadly, the more important features of the versatile systems, apparatuses and accompany methods in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carries out in various ways. Also, it is to be understood that the phraseology and terminology employed herein are for the purpose of description and should not be regarded as limiting.

These, together with other objects of the invention, along with the various features of novelty, which characterize the invention, are pointed out with particularity in the claims annexed to and forming a part of this disclosure. For a better understanding of the interactive systems, apparatuses and accompany methods, the operating advantages and the specific objects attained by usage, reference should be made to the accompanying drawings and descriptive matter in which there are illustrated preferred embodiments of the invention.

### BRIEF DESCRIPTION OF THE DRAWINGS

The invention may be more completely understood in consideration of the following detailed description of various embodiments of the invention in connection with the accompanying drawings, in which:

FIG. 1 illustrates a perspective view one embodiment of the vaporizing device illustrating the vaporization or atomization portion and electronics portion;

FIG. 2 illustrates an exploded view of the atomizer liquid reservoir and the main airway regions of the atomizer portion;

FIG. 3A illustrates an exploded view of the upper region of the atomization portion in a disassembled state, including the mouthpiece and the coil;

FIG. 3B illustrates an exploded view of the disassembled atomization portion, including the liquid reservoir;

FIG. 3C illustrates an exploded view of the disassembled atomization portion, including the main airway, the atomizer, the airflow ring and heat transfer component;

FIG. 4A illustrates the connection means between the electronics housing and the atomization portion and further comprises a heat transfer component of the top end of the electronics housing;

FIG. 4B illustrates the connection means between the electronics housing and the atomization portion and further comprises a heat transfer component of the top end of the electronics housing;

FIG. 4C illustrates the connection means between the electronics housing and the atomization portion and further comprises a heat transfer component of the top end of the electronics housing;

FIG. 5A illustrates an exploded view of the atomization portion components including the cotton pad and main airway;

FIG. 5B illustrate an exploded view of the atomizer components including the metal mesh and the metal coil which is attached to the heat transfer component;

FIG. 6A illustrates a perspective view of an embodiment of the present system utilizing a facemask for attachment to the atomization portion to form an occlusive seal with the facial surface of the user;

FIG. 6B illustrates a bottom view of an embodiment of the present system utilizing a facemask for attachment to the atomization portion to form an occlusive seal with the facial surface of the user;

FIG. 7 illustrates a top view of the contents of the electronics housing including a circuit board or electronics chip, a screen, an ON button, a USB port, a battery, and electronic connection to the heating member;

FIG. 8 illustrates a perspective view of an embodiment of the face mask nasal adapter heating element, an atomizer, and a removably attached face mask;

FIG. 9 illustrates a side view environmental view of the embodiment of the face mask nasal adapter of FIG. 8 illustrating the face mask in use occlusively sealed against the face of a user;

FIG. 10 illustrates a side view environmental view of an additional embodiment of the face mask nasal adapter illustrating the face mask in use with an additional embodiment of the delivery system wherein the face mask is occlusively sealed against the face of a user;

FIG. 11 illustrates a front perspective environmental view of an additional embodiment of the face mask illustrating the face mask in use with an additional embodiment of the delivery system wherein the face mask is occlusively sealed against the face of a user through the use of a raised;

FIG. 12 illustrates a side view of the nasal adapter or face mask embodiment of FIG. 11 further illustrating the raised, arcuately disposed facial contact surface oriented for oral usage.

FIG. 13 illustrates a side view of the nasal adapter or face mask embodiment of FIG. 11 further illustrating the features wherein the face mask may comprise a substantially conical area and a raised, arcuately disposed facial contact surface.

FIG. 14 illustrates a side view of one embodiment of a cartridge to be utilized within the vaporizing system of FIG. 8, including an exploded view of the incorporated drip-tip check valve or one-way valve.

FIG. 15 is a sectional plan view of an additional embodiment of the vapor delivery system, particularly illustrating one embodiment of cartridge which includes an absorbent sponge and an atomizer.

FIG. 16 illustrates an alternative embodiment of the vaporizing device in which a separation means divides the atomizer an upper chamber and a lower chamber for generating additional vapor.

FIG. 17 illustrates the battery charging system including the charger, the charging, the USB connector and the indicator light.

### DETAILED DESCRIPTION OF THE INVENTION

The detailed description set forth below is intended as a description of presently preferred embodiments of the apparatus and does not represent the only forms in which the present apparatus may be construed and/or utilized. The description sets forth the functions and the sequence of the steps for producing the system and accompanying apparatus. However, it is to be understood that the same or equivalent functions and sequences may be accomplished by different embodiments also intended to be encompassed within the scope of the invention.

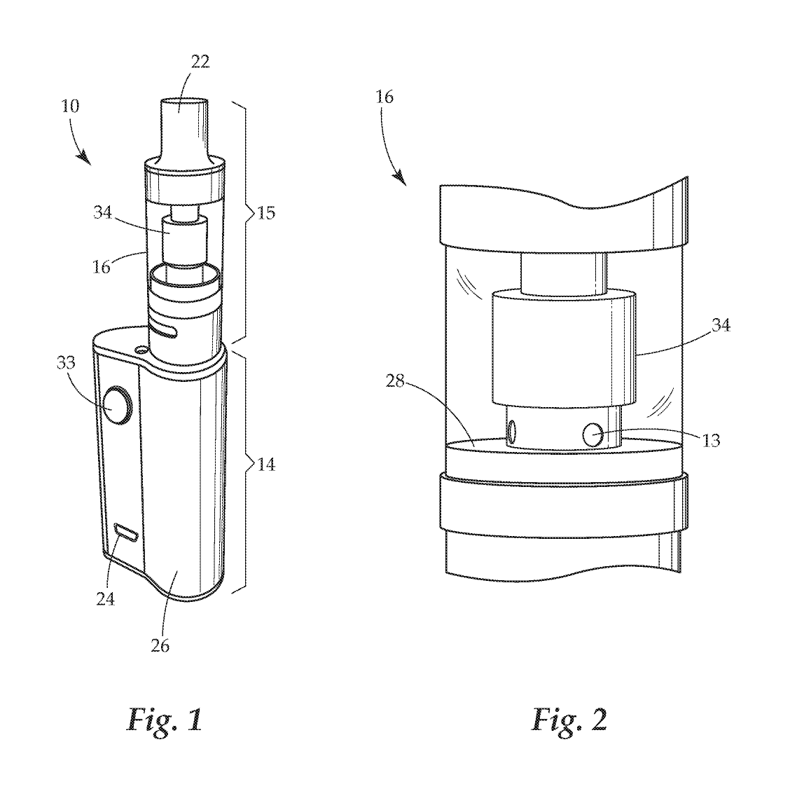

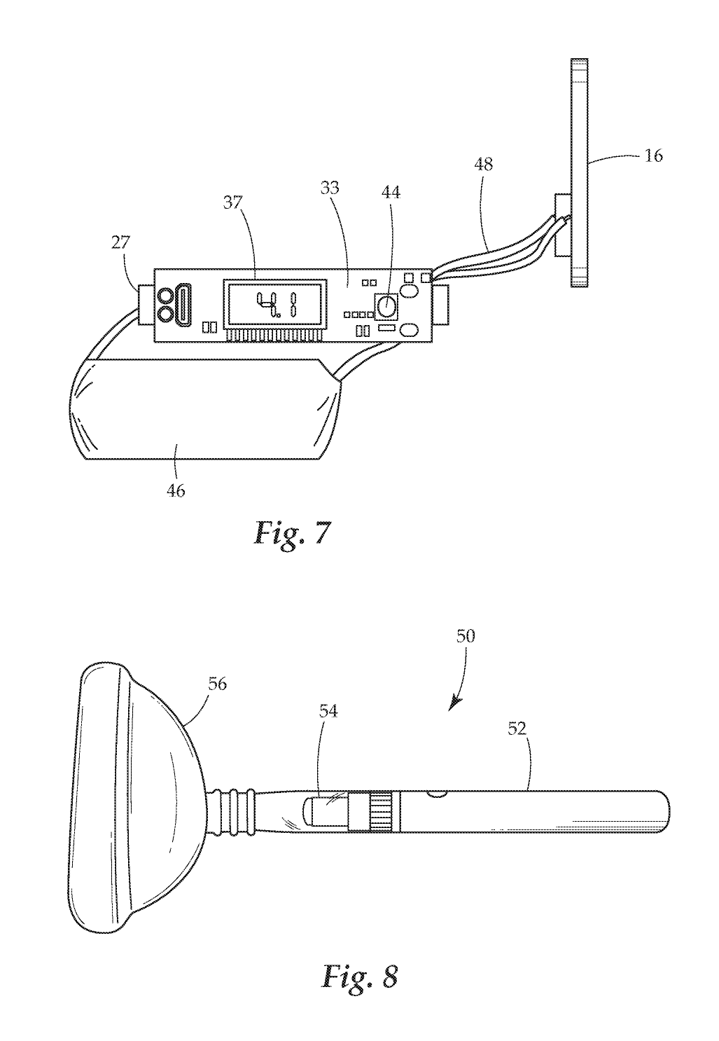

FIG. 1 illustrates a perspective view one embodiment of the vaporizing device 10, which includes a vaporization or an atomization portion 15 and electronics portion 14. In one embodiment, the atomization portion 15 may further comprises a mouthpiece 22, liquid reservoir 16 and atomizer 28. In an additional embodiment, the electronics portion 15 further comprises an outer shell 26, USB port 24, and ON Button 33. In one embodiment, the mouthpiece 22 may comprise a top filling mouthpiece. The integrated circuit (IC) or microprocessor 27 (shown in FIG. 7) releases a signal to activate and deactivate said power source, through the integrated circuit (IC), in this case the microprocessor 27.

The mouthpiece 22 is under negative pressure and a quantity of air pressure thus generated releases an actuating signal to microprocessor 27 to activate the power source and vapor is generated when the liquid makes contact with the atomizer permitting air to be drawn past a cartridge for inhalation by the user at the mouthpiece.

FIG. 2 illustrates an exploded view of the atomizer liquid reservoir 16 and the atomizer 28. The atomization portion 14 further comprises a set of atomizer liquid inlets 13. In one embodiment, the user fills the liquid reservoir 16 with the liquid to be vaporized and liquid slowly trickles into the atomizer 28 through the set of atomizer liquid inlets 13.

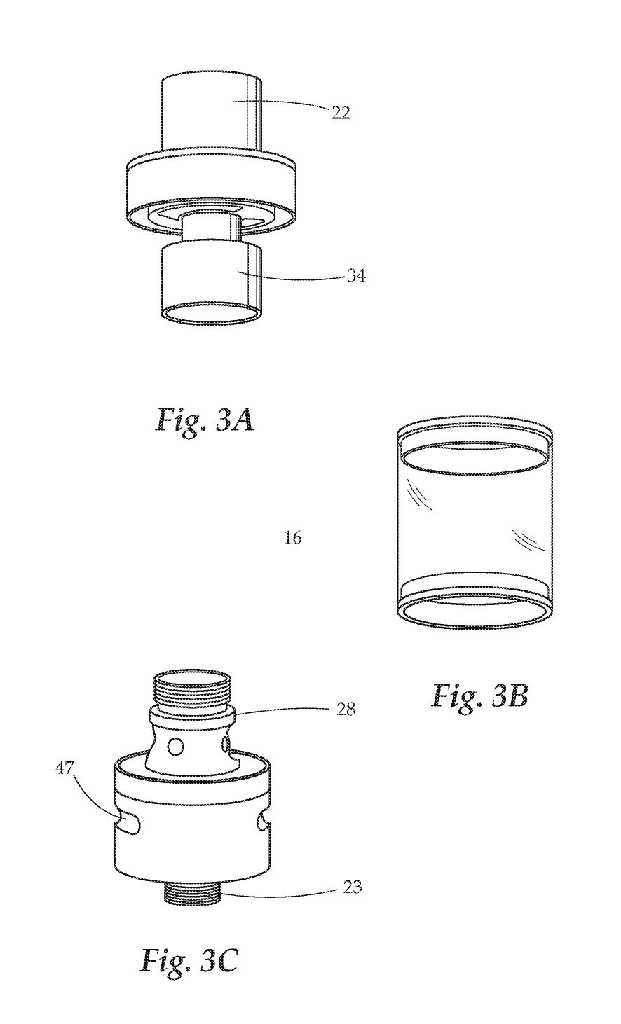

FIGS. 3A-3C illustrate a set of exploded views of the disassembled atomization portion, including the mouthpiece 22, coil 34, the liquid reservoir 16, the main airway 44, and the heat transfer component 23.

FIG. 3A illustrates an explode view of the upper section of the disassembled atomization portion, including the mouthpiece 22 and the coil 34. A key element to the creation of the nano-mist or nano-particles focuses the utilization of high coil temperatures under dry, wet-through-wick, and full-wet conditions. Many factors, such as coil material and resistance, battery voltage, atomizer/wick design, e-liquid composition and fill level, and vaping topography (puffing time, interval, or volume) may affect the operating temperature of e-cigarettes. The liquid reaches a temperature of roughly 100° C.-250° C. or greater within the atomizer 28 to create an aerosolized vapor and the product of such conditions respectively create droplet sizes varying from 50 nano-meters to 400 nano-meters.

Numerous recent studies have indicated that delivery of certain applicable substances through nasal cavities has proved more advantageous than through conventional oral intake methods. In line with these studies, the nano-particles created by the present system, due to the high temperature vaporization, are specifically designed and suited for such activity in order to directly reach the bronchioles are some of the smallest airways in your lungs. Thus, the nano-particles are inhaled and pass through tiny ducts from the bronchioles into elastic air sacs—or alveoli.

FIG. 3B illustrates an explode view of the median section of the disassembled atomization portion, including the liquid reservoir 16.

FIG. 3C illustrates an exploded view of the lower section of the disassembled atomization portion, including the atomizer 28, the airflow ring 47 and heat transfer component 23.

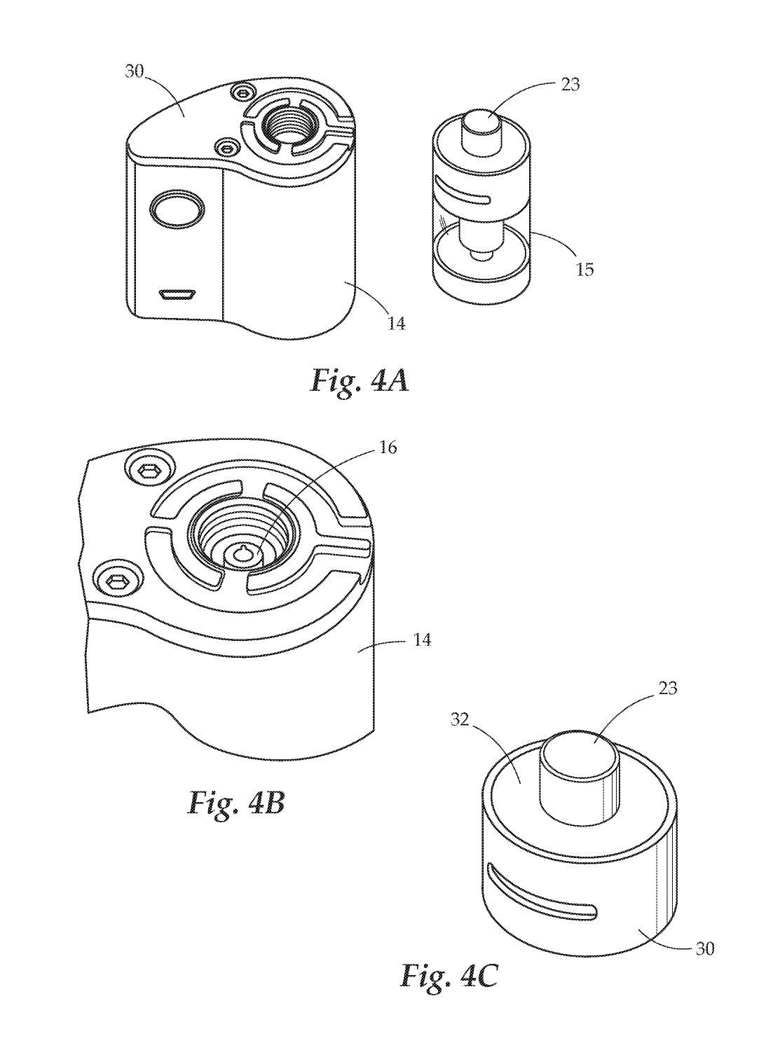

FIGS. 4A-4C illustrates the connection means between the electronics housing 14 and the atomization portion 15. The upper portion 30 of the electronics housing 14 further comprises a heat transfer component 23, which becomes hot by means of a battery and electric circuit board located within the electronics housing 14 (seen in FIG. 7).

The bottom portion 32 of the atomization portion 15 further comprises a heating member 16, which mates to the heat transfer component 23 by means of threading. The heating member 16 transfers heat through the heat transfer component 23, thereby providing heat for the atomization process.

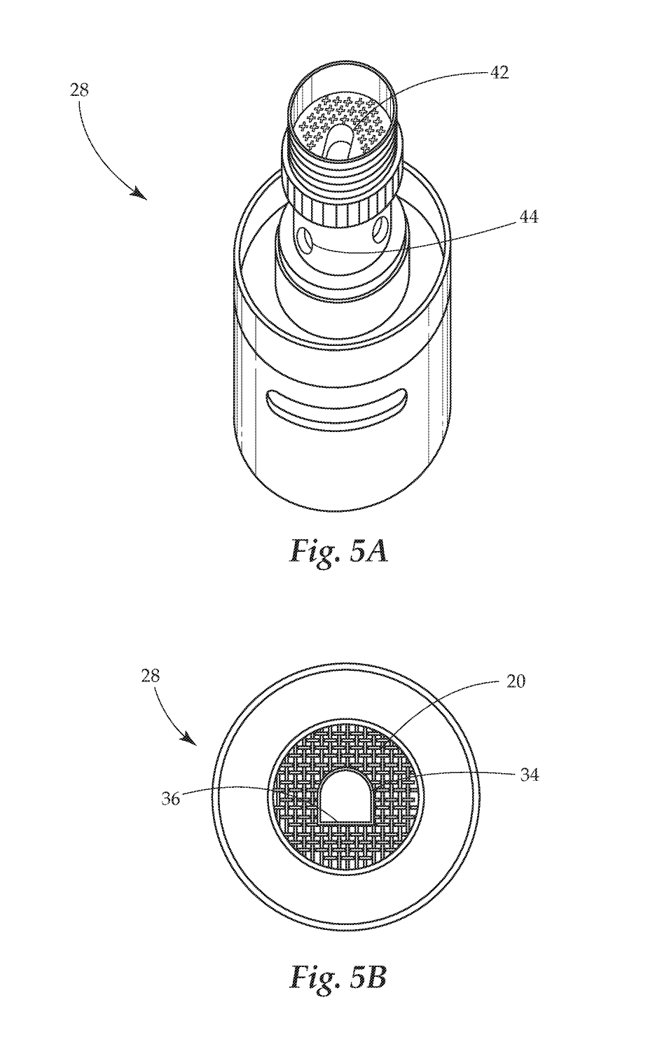

FIGS. 5A and 5B illustrate an exploded view of the atomizer 28, which further comprises a metal mesh 20 and cotton pad 42. Inside of the atomizer 28, there is a tank and metal coil 34, which is attached to the heat transfer component 23. The cotton pad 22 soaks up liquid, which secretes through the atomizer liquid inlet 13. The tank and metal coil 34 contacts the soaked cotton pad 42 and vaporizes the liquid stored in the cotton pad 42. As the user inhales, the vapor moves up through the lower airway 36, into the main airway 44, through the mouthpiece 22, and into the user's mouth.

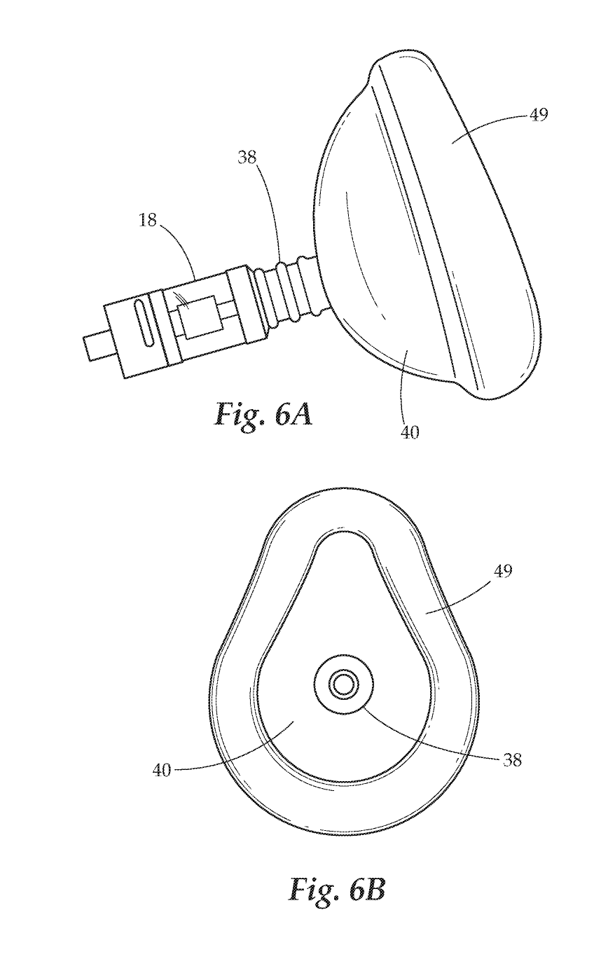

FIGS. 6A and 6B illustrate perspective views of a facemask 40 which may be removably attached or permanently fixed to the atomization portion 15. In one embodiment, the facemask 40 may comprise a substantially conical area and a compliant or flexible connector or coupling mechanism for removable attachment to the vaporizing system. The facemask further comprises a raised, arcuately disposed facial contact surface 49, which creates an airtight, occlusive seal between the user's face and the facemask 40.

FIG. 6A illustrates a side perspective view of an embodiment of the present system utilizing a facemask 40 for attachment to the atomization portion 15 and the mouthpiece 22 to form an airtight, occlusive seal between the user's face and the facemask 40.

FIG. 6B illustrates a bottom view of an embodiment of the present system utilizing a facemask 40 for attachment to the atomization portion 15 and the mouthpiece 22 to form an occlusive seal with the facial surface of the user. The facemask 40 further comprises a raised, arcuately disposed facial contact surface 49, which assists in the creation an airtight, occlusive seal between the user's face and the facemask 40.

In one embodiment, the facemask attaches to the mouthpiece 22 by means of a compliant or flexible connector 38, which remains in place due to the friction between the compliant or flexible connector 38 and the surface of the mouthpiece 22. The compliant or flexible connector 38 may contain a ringed or threaded outer portion in order to assist the user in the installation and removal of the compliant or flexible connector 38.

In one embodiment, the facemask 40 may further comprise a flexible, elastomeric material, capable of retaining an airtight connection with a variety of surface textures for adaptation to differing vapor dispensing systems as illustrated herein.

FIG. 7 illustrates a top view of the contents 42 of the electronics housing 4. The contents 4 further comprise a microprocessor 27, screen 37, ON button 33, USB port 24, battery 46, and electronic connection 48 to the heating member 16. When the user presses the On button 33, the heating member 16 generates heat.

FIG. 8 illustrates a side perspective view of an additional embodiment of a vaporizing system 50 with a face mask nasal adapter. Herein, a user 58 is utilizing the nasal adapter with an internal battery device and heating element 52, an atomizer 54, and a removably attached face mask 56.

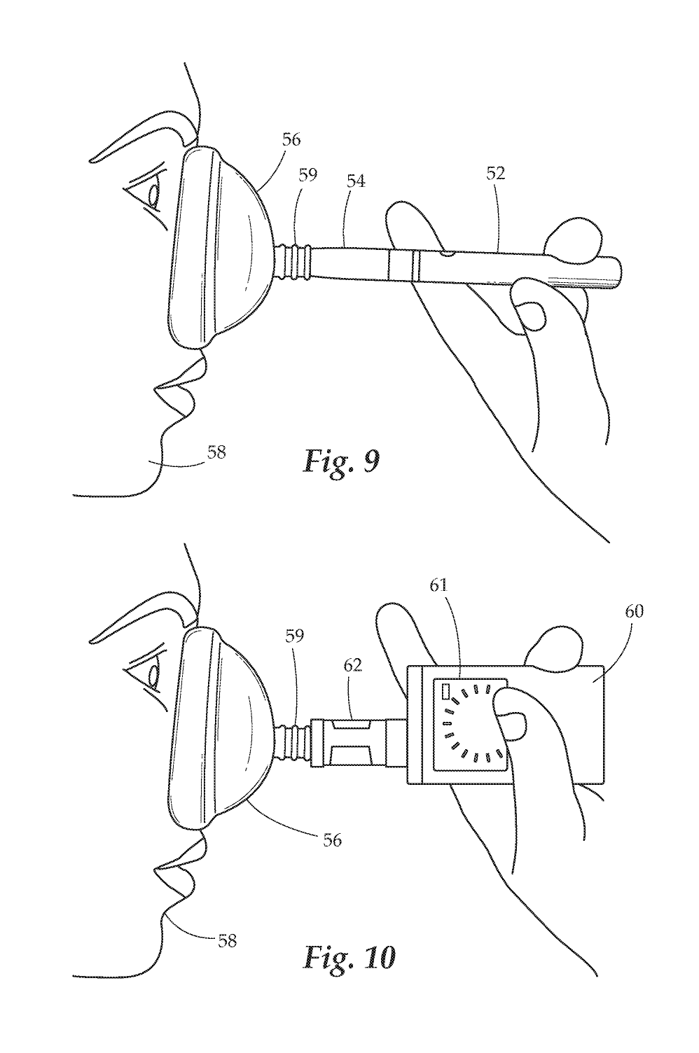

FIG. 9 illustrates a side view environmental view of the embodiment of the nasal adapter or face mask 56 illustrating the face mask 56 in use occlusively sealed against the face of a user 58.

FIG. 10 illustrates a side view environmental view of an additional embodiment of the nasal adapter or face mask 56 illustrating the face mask 56 in use with an additional embodiment of the delivery system wherein the face mask 56 is occlusively sealed against the face of a user 58. In operation the user 58, utilizes the face mask 56 in conjunction with a digital vaporizer system 60, wherein a digital read out electronic portion 61 and atomizer portion work in conjunction to allow the user to adjust temperature, vaporization, and other qualities for a customized use depending on the substance used. When in use, a user places the face mask over his nose area, activates the atomizer, and breathes in the released vapor.

Further illustrated is the compliant or flexible connector 59 which may include grip ribs or grip threads 57 for ease of attachment and removal, and the atomizer 54 utilized for this embodiment.

In a preferred embodiment, the face mask mechanism 16 provides a one-way valve, as illustrated in FIG. 13, which allows for a dispensing of mist and vapor that would be inhaled by the user 58. In one embodiment, the battery device is removable so that when exhausted, a fresh dry battery may be substituted.

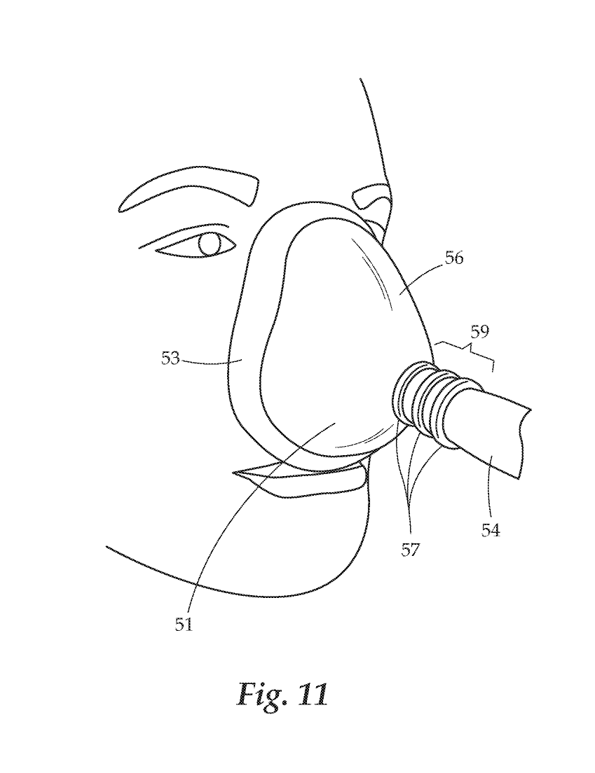

FIG. 11 illustrates a front perspective environmental view of an additional embodiment of the nasal adapter or face mask 56 illustrating the face mask in use with an additional embodiment of the vapor delivery system, wherein the atomizing portion 54 of the vapor delivery system is illustrated. This perspective view more clearly illustrates how the face mask 56 matches the contour of the user face as well as the outward extension of the nasal cavity of the user.

The face mask 56 may comprise a substantially conical area 51 and a raised, arcuately disposed facial contact surface 53. In numerous embodiments, the raised, arcuately disposed facial contact surface 53 may be composed of a more malleable and substantially less dense or compliant materials than that of the conical area 51. Thus, the compliant material of the facial contact surface 53 will provide an occlusive seal against the face of a user and the raised, arcuately disposed facial contact surface 53 is designed to match the contour of area surrounding the human nasal area to provide for minimal gapping with the occlusive seal.

The facemask 56 may be composed of numerous applicable polymers and in one embodiment silicone may be utilized. Thus, in the manufacturing processes, the conical area 51 will be molded, pressed or extruded to be denser and much less flexible than the thin, facially form fitting material of the facial contact surface 53.

Furthermore, the facemask 56 may also include a compliant or flexible connector or coupling mechanism 59 for removable attachment to the vaporizing system 54. The facemask 40 further comprises a raised, arcuately disposed facial contact surface 49, which creates an airtight, occlusive seal between the user's face and the facemask 40.

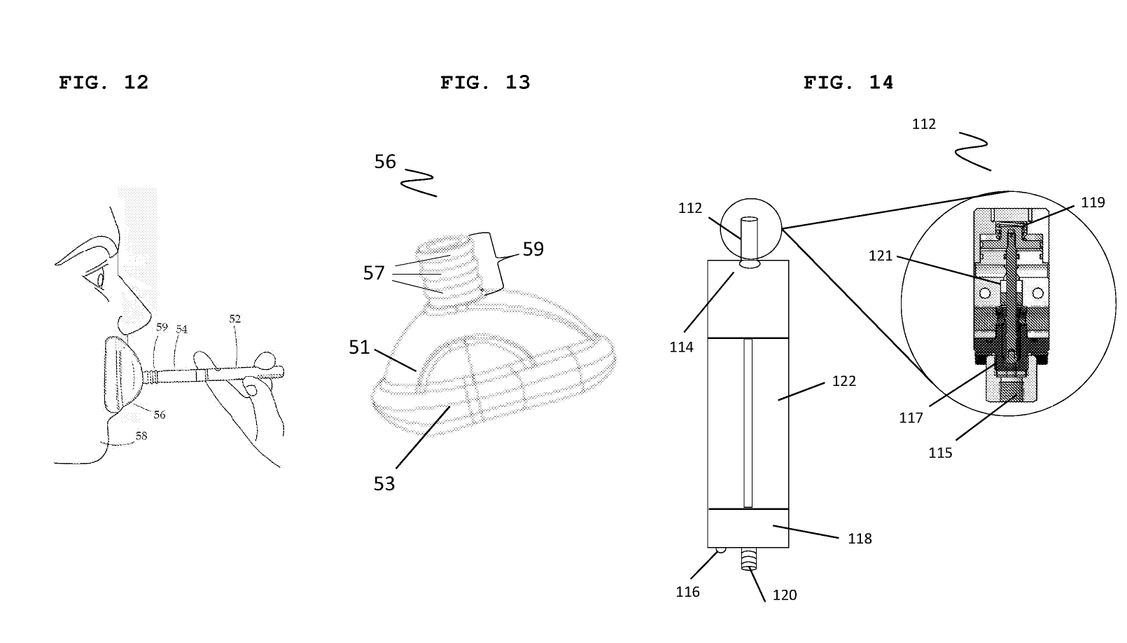

FIG. 12 illustrates a side view of the face mask 56 of FIG. 11 further illustrating the features wherein the face mask 56 may comprise a substantially conical area 51 and a raised, arcuately disposed facial contact surface 53. In this embodiment, illustrated the raised, arcuately disposed facial contact surface oriented for oral usage.

Again the raised, arcuately disposed facial contact surface 53 may be composed of a more malleable and substantially less dense or compliant materials than that of the conical area 51. Thus, the compliant material of the facial contact surface 53 will provide an occlusive seal against the face of a user and the raised, arcuately disposed facial contact surface 53 is designed to match the contour of area surrounding the human nasal area to provide for minimal gapping with the occlusive seal. The facemask 56 may also compliant or flexible connector or coupling mechanism 59 for removable attachment to a vaporizing system.

FIG. 13 illustrates a side view of the nasal adapter or face mask 56 of FIG. 11 further illustrating the features wherein the face mask 56 may comprise a substantially conical area 51 and a raised, arcuately disposed facial contact surface 53. Again the raised, arcuately disposed facial contact surface 53 may be composed of a more malleable and substantially less dense or compliant materials than that of the conical area 51. Thus, the compliant material of the facial contact surface 53 will provide an occlusive seal against the face of a user and the raised, arcuately disposed facial contact surface 53 is designed to match the contour of area surrounding the human nasal area to provide for minimal gapping with the occlusive seal. The facemask 56 may also compliant or flexible connector or coupling mechanism 59 for removable attachment to a vaporizing system of choice.

FIG. 14 illustrates a side view of one embodiment of a cartridge 122 to be utilized within the vaporizing system 50 of FIG. 8, including an exploded view of the incorporated check valve or one-way valve 110. The cartridge 122 is fully-sealed and further comprises a spring-loaded valve 114, a fill valve 116, and an atomizer 114 located at the base of the cartridge with a threaded fastening mechanism 120. Also illustrated is a drip-tip with a one-way valve 110 that facilitates flow of oil onto the vaporizing surface, which comprises a heated surface such as a coil, metal plate or other heating means.

FIG. 14 further illustrates an exploded view of the spring-loaded valve, which further comprises an inlet tract 115, disc 117, spring 119, spring rod 123, and seal 121. The spring-loaded valve 114 is engaged when the user orally creates the anterior end 172 of the vaporization device 158 through the mouth piece, as shown FIG. 14. The pressure drop caused by this sucking action causes the spring rod 123 to push up on the spring 119 and release a controlled amount of liquid into the atomizer 114 through the inlet tract 115.

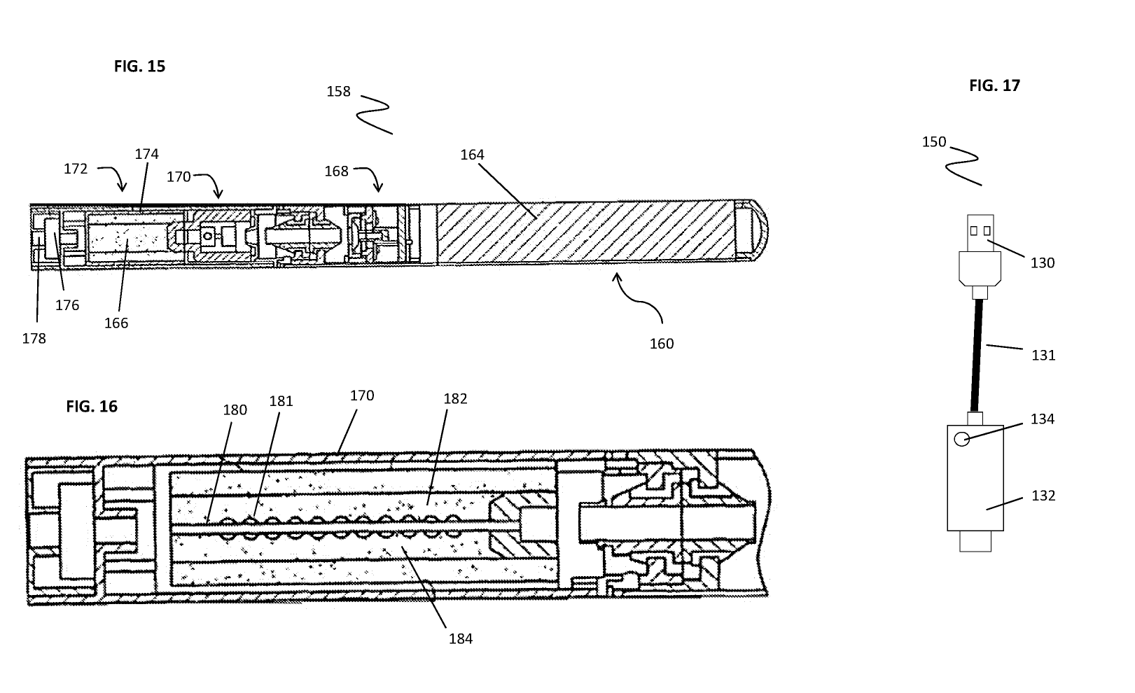

FIG. 15 is a sectional plan view of an embodiment of the vapor delivery system. The system includes an outer housing 158 which includes a proximal end 172 and a distal end 160. The proximal end 172 of the housing is design to be oriented toward the user's mouth. The distal end 160 of the housing 158 is proximate to the battery 164. The cartridge 174 includes an absorbent sponge 166, atomizer 14 and a liquid supplying bottle. The proximal end 172 of the housing 158 includes the mouthpiece 178 and a filter 176.

The filter 176 further comprises a means of preventing liquid from leaving the proximal end of the housing while facilitating the diffusion of vapor into the user's mouth. The mouthpiece 178 attaches to the proximal end. When the user inhales, the mouthpiece 178 experiences negative pressure that activates an actuating signal to an IC switch 168 to activate the power source. This system may also include a smart chip and control panel as and a lithium ion rechargeable battery 12 as a power source as shown in FIG. 1. Vapor is generated when the heated liquid makes contact with the atomizer 14.

FIG. 16 illustrates an alternative embodiment of the vaporizing device in which a separation means 180 divides the atomizer 14 an upper chamber 182 and a lower chamber 184 for generating additional vapor. The vaporizer further comprises a metal coil 181, which is heated to vaporize oil in the upper chamber 182 and lower chamber 184.

FIG. 17 illustrates the battery charging system 150. To charge the battery, remove the cartridge, 122 containing the liquid. Once the cartridge has been properly removed, gently screw the cartridge into the charger 132. With the battery attached to the charging cable 131, plug the charging cable 131, which contains a USB end 130, into any USB slot or use a wall plug and connect it to a 110 Volt electric outlet. There is an indicator light 134 on the charging cable that will glow red while charging. Once the battery 12 is done charging, the indicator light 134 will glow green.

In numerous embodiments, a cylindrical mechanism or an external tube made preferably the size, color and form of a cigarette, such tube having an outer end portion and an inner end portion, subdividing the interior space of the external tube is an internal shoulder which is preferably closer to the outer end portion than to the opposite inner end portion, thus dividing the tube into relatively short and substantially longer chambers. In some embodiments, the battery device is removable so that when exhausted, a fresh dry battery may be substituted.

The tube is supplied at the inner end portion with internal threads or some other means of connection for a mouthpiece as later described. In the outer end portion is detachably fitted a flavor cartridge of some suitable absorbent material, preferably having longitudinal spaced passages there through of a small diameter. The cartridge is abutted against the outer edge of the internal shoulder and is of an external diameter to fit snugly into the outer end portion so that it will be held therein by friction or other suitable means.

Within the more forward chamber of the outer tube is received a tubular liner preferably of insulating material and having an internal wall of a form and character to tumble the air or create turbulence therein. This internal wall may be spiraled or rifled as indicated. A mouthpiece is affixed to the inner end portion of the tube in any appropriate manner, preferably detachably as by external threads on the hollow shank of the mouthpiece which mate with the internal threads in the inner end portion of the outer tube.

The free outer end of the hollow shank, when fitted home in the outer tube, will encounter and push the insert against the inner wall of the internal shoulder thus holding the insert immovably in place.

The hollow shank will preferably have an outstanding shoulder forwardly of the threads to engage the inner end of the tube in the completely assembled position of the mouthpiece relatively to the outer tube.

A cross-section through the hollow shank portion of the mouthpiece, a spider formation is shown providing air draft spaces between the hollow shank and an inner ring spaced inwardly from the hollow shank and connected therewith by radial arms. The air draft spaces communicate with the suction orifice of the mouthpiece at the inner end and at the outer end with the space circumscribed by the inner wall of the tubular insert.

Within the inner ring is a threaded electric socket and forwardly thereof a battery cavity for detachably receiving a battery having an inner contact and an outer contact with a contact strip between the inner contact and the socket.

The heating element is preferably a vacuum tube or bulb having a screw plug for detachable engagement with the socket. The screw plug has an end contact adapted to close against the outer battery contact. The bulb or tube, similar to a light bulb, is preferably elongated and of a diameter to fit within the insert in such manner heating passage throughout the length of the bulb and around the complete circumference of the bulb in assembly, the flavor cartridge can be introduced and removed without regard to the other units of the device.

Before the mouthpiece is assembled to the external tube, the liner is slid through the open inner end portion of the tube until the outer end of the liner encounters the internal shoulder. The bulb will be mounted to the socket while the mouthpiece is detached from the tube whereupon the bulb may be introduced into the insert as the mouthpiece is put into place and rotated to effect attachment of the mouthpiece to the outer tube. The final home position of the parts is indicated in which the hollow shank engages the inner end of the insert while the outer end of the insert is abutted against the internal shoulder.

When the bulb is assembled to the socket its tip end will engage the inner battery contact and complete the circuit through the filament of the bulb by the contact strip. Thus the bulb will be illuminated or energized before assembly to the external tube.

The battery is removable so that when exhausted, a fresh dry battery may be substituted, access to the same being had by first unscrewing the mouthpiece and withdrawing the bulb and subsequently removing the bulb from its socket.

The insert is preferably of a ceramic material. By the act of inhalation through the mouthpiece, air from the ambient atmosphere is drawn in through the passages of the cartridge which has been impregnated with suitable flavoring material which is picked up by the inhaled air and such air then passes into the heating chamber and is caused to flow around the heating bulb, the heated and flavored air finally passing through the mouth piece into the mouth and also, if desired, into the lungs.

An air circulation or recirculation system may be incorporated in order to cool the outer surface of the mechanism. The cartridge may contain a drip tip and a one-way valve to prevent the spread of disease if reused. The cartridge may be reusable or disposable, as may well be the tip mechanism.

The impregnation will be by a harmless flavored chemical compound. As suggested, such compounds may be solutions ranging from slightly mentholated water to a solution which would simulate artificially the flavor of menthol, or other lung soothing or cleansing substance or mixture.

A list of some herbals, nutraceuticals and configuration (which do not include regulatory requirements) that may be administered through the instant system include, but are not limited to the following:

1) Lung Cleansing Kits (including the active ingredients along the lines of menthol and other akin to Vics VapoRub®);
2) Weight Control systems (Active ingredients of Hydroxicut®
3) Sleep Aid (Melatonin, diphenhydramine and other OTC drugs);
4) Anxiety, depression, relaxant (Valerian Root Extract with or without Bay Leaf Extract);
5) Energy Formulations used in HealthCare®, sold in Whole Food® stores;
5) CoQ10® (High priced formulations sold in GNC®, etc.);
6) Pain (Naproxen®, Ibuprofen® other NSAIDs);
7) Sleep Aid (melatonin, diphenhydramine and other OTCs);
8) Migraine relief nutraceuticals or herbals.

The system may further comprise variations such as the use of lavender type extracts for the purposes of:
a. preventing panic attack and anxiety;
b. calming effect aids relaxation;
c. reducing stress; and,
d. relieving pain from tension headache.

Thus, herein is presented a lung cleaning system driven by utilization of nano-particles, in conjunction with a containment system such as a mask, to ensure delivery of the nano-particles. The system is illustrated by example in the figures, and throughout the written description. It should be understood that numerous variations are possible, while adhering to the inventive concept. Such variations are contemplated as being a part of the present apparatus and set of accompanying systems.

It should be understood that numerous variations are possible, while adhering to the inventive concept. Such variations are contemplated as being a part of the present invention.

What is claimed is:

1. A lung cleansing system comprising:
a vaporizing system comprising:
a mouthpiece;
a liquid reservoir and vaporizing liquid;
a main airway; and
an atomization portion wherein the atomization portion further comprises;
an atomizer;
a heating coil; and
heat transfer component;
an electronic portion comprising:
an outer shell;
a microprocessor;
a USB port; and
an ON Button; and
a face mask in communication with the mouthpiece.

2. The lung cleansing system of claim 1 wherein the face mask further comprises a raised, arcuately disposed facial contact surface.

3. The lung cleansing system of claim 1 wherein the face mask comprises a compliant connector.

4. The lung cleansing system of claim 3 wherein the compliant connector comprises a set of grip threads.

5. The vaporized cleaning lung system of claim 1, wherein the vaporizing system further comprises a drip tip one way valve.

6. The lung cleansing system of claim 1 wherein the mouthpiece comprises a top filling mouthpiece.

7. The lung cleansing system of claim 1 wherein the heating element hats the liquid to a temperature range from 100° C.-250° C.

8. The lung cleansing system of claim 1 wherein the system heats vapor to creates nano-particles in a size range from fifty nano-meters to four hundred nano-meters.

9. The lung cleansing system of claim 1 wherein the face mask comprises a raised, arcuately disposed facial contact surface disposed to match the contour of the area surrounding the human nose to provide an occlusive seal.

10. A vaporized lung cleansing system comprising:
a main body for enclosing said vaporized lung cleansing system, said body having an anterior end and a distal end;
a cartridge, comprising an absorbent sponge;
an atomizer and a liquid supplying bottle;
a power source;
a heating element within the distal end of the body for transferring heat to the liquid;
a mouthpiece on the anterior end of the body for drawing atmosphere air by inhaling through the anterior end of the body including the cartridge and through the space between the heating element;
an integrated circuit (IC) switch for releasing a signal to activate and deactivate said power source, wherein the mouthpiece is under negative pressure and a quantity of air pressure thus generated releases an actuating signal to integrated circuit (IC) to activate the power source and vapor is generated when the liquid makes contact with the atomizer permitting air to be drawn past a cartridge for inhalation by the user at the mouthpiece; and
a removably attached face mask.

11. The vaporized lung cleansing system of claim 10 wherein the heating element hats the liquid to a temperature range from 100° C.-250° C.

12. The vaporized lung cleansing system of claim 10 wherein the atomization portion heats vapor to creates nano-particles in a size range from fifty nano-meters to four hundred nano-meters.

13. The vaporized lung cleansing system according to claim 10, wherein said atomizer includes an upper chamber and lower chamber.

14. The vaporized lung cleansing system of claim 10 wherein the system delivers plain menthol to cleanse lungs.

15. The vaporized cleaning lung system of claim 10, wherein the mouthpiece has a drip tip comprising a one way valve.

16. The vaporized cleansing lung system of claim 1 wherein the cartridge is removable.

17. A method of delivering vapor to the lungs comprising the steps of:
providing a vaporized lung cleansing system having a main elongated cylindrical body for enclosing said vaporized lung cleansing system and having an anterior and distal end and further comprising:
a cartridge;
an absorbent sponge;
an atomizer;
a heating element;
a mouthpiece;
a removably attached face mask;
a power source;
an IC switch for releasing a signal to activate and deactivate the power source; and;
injecting a quantity of substances into the main elongated cylindrical body;
generating vapor is generated when a quantity of liquid makes contact with the atomizer;
heating the vapor to a range of 100° C.-250° C. to creates nano-particles in a size range from fifty nano-meters to four hundred nano-meters;
routing the nano-particles through the mouthpiece by creating negative pressure and generating a quantity of air pressure to release an actuating signal to an IC switch; and
routing the nani-particles from the mouthpiece to face mask.

18. The method of delivering vapor to the lungs of claim 17, wherein substances are selected from the group consisting of lavender oil, caffeine, menthol, eucalyptus, glycerol beads, water, alcohol, nutraceuticals, herbals, prescription and non-prescription drugs, vitamins, vaccines, anti-pyretics, narcotic and non-narcotic pain relievers, diabetes medications, cystic fibrosis medications, obesity medications and anti-ageing medications.
